(19) United States
(12) Patent Application Publication
(10) Pub. No.: US 2026/0097213 A1
(43) Pub. Date: Apr. 9, 2026

(54) OPTIMAL VECTORS FOR ELECTRICAL THERAPIES

(71) Applicant: Stryker Corporation, Portage, MI (US)

(72) Inventors: David J. Linville, Redmond, WA (US); Tyson G. Taylor, Bothell, WA (US); Rose Tingwei Yin, Bellevue, WA (US); Fred W. Chapman, Newcastle, WA (US); Robert G. Walker, Seattle, WA (US); Ryan William Apperson, Bothell, WA (US); Rockland W. Nordess, Kirkland, WA (US); Reza Sharif, Lake Forest, WA (US); Dennis Changmin Sohn, Seattle, WA (US); Michelle Bohner Robershotte, Kirkland, WA (US); Trenton Roeber, Carnation, WA (US)

(73) Assignee: Stryker Corporation, Portage, MI (US)

(21) Appl. No.: 19/352,274

(22) Filed: Oct. 7, 2025

Related U.S. Application Data
(60) Provisional application No. 63/705,466, filed on Oct. 9, 2024.

(51) Int. Cl. A61N 1/37; A61N 1/04

(52) U.S. Cl. CPC A61N 1/3702; A61N 1/046; A61N 1/0476

(57) ABSTRACT

An example method includes identifying feedback from electrical signals output along multiple vectors to multiple electrodes configured to be disposed on skin of a subject. The method further includes selecting, among the multiple vectors, an optimal vector by analyzing the feedback; and identifying, among the multiple electrodes, a first electrode and a second electrode associated with the optimal vector. In response to identifying the first electrode and the second electrode associated with the optimal vector, a recommendation to administer an electrical shock to the first electrode and the second electrode is output; or the electrical shock is output to the first electrode and the second electrode.

### CROSS-REFERENCE TO RELATED APPLICATION

This application claims the priority of U.S. Provisional App. No. 63/705,466, which was filed on Oct. 9, 2024 and is incorporated by reference herein in its entirety.

### BACKGROUND

Particular heart arrhythmias, such as ventricular fibrillation (VF) and pulseless ventricular tachycardia (VT), are deadly if untreated. An individual with one of these arrhythmias can be treated by administering an electrical shock to the individual's heart. This treatment is known as defibrillation. The arrhythmias that are treatable by defibrillation are known as shockable arrhythmias.

Unfortunately, some instances of shockable arrhythmias are resistant to conventional defibrillation therapies. For instance, an individual is determined to have refractory VF if they have VF that continues through the administration of an electrical shock. Recently, vector change and double sequential defibrillation (DSD) (also referred to as “double sequential external defibrillation” or “DSED”) have been proposed as an alternative to single-shock defibrillation. A DSD therapy, for instance, can be administered by discharging two electrical shocks to an individual's heart, rather than one. Researchers have suggested that DSD administration can increase survivability of refractory VF. Cheskes et al., 387 N. Engl. J. Med. 1947 (2022).

### BRIEF DESCRIPTION OF THE DRAWINGS

FIG. 1 illustrates an example environment for optimizing defibrillation treatments based on vector selection and vector change.

FIG. 2 illustrates example signaling for selecting an optimal vector for administering a therapy to a subject.

FIG. 3A illustrates an example electrode accessory suitable for automated vector change and selection.

FIG. 3B illustrates an example environment of a connector configured to facilitate the administration of electrotherapies at different vectors.

FIG. 4 illustrates an example process for selecting an optimal vector for administration of a therapy to a subject.

FIG. 5 illustrates an example process for selecting an optimal vector based on transthoracic impedance.

FIG. 6 illustrates an example of an external defibrillator configured to perform various functions described herein.

FIGS. 7A and 7B illustrate examples of environments and timing related to administering a multi-shock (e.g., DSD) therapy. FIG. 7A shows an environment configured to administer the multi-shock therapy. FIG. 7B shows a timing relationship of multiple shocks administered in the multi-shock therapy.

### DETAILED DESCRIPTION

Various implementations described herein relate to selecting optimal vectors for defibrillating shocks. In some cases, a shockable arrhythmia (e.g., VF) is resistant to defibrillation when electrical shocks are administered to non-optimal vectors. These non-optimal vectors include pathways between electrodes that do not substantially pass through the heart. Accordingly, in cases where a defibrillator is configured to administer electrical shocks to a subject along a single shock vector, and that shock vector is a non-optimal vector, it may be unable to effectively treat a shockable arrhythmia of the subject.

In various implementations of the present disclosure, at least one defibrillator is configured to be connected to external electrodes disposed along multiple shock vectors. In some cases, multiple electrodes are integrated into a single electrode pad that is disposed on the skin of the subject. The defibrillator(s), in various cases, outputs electrical signals along the multiple shock vectors and detects feedback based on the electrical signals. Based on the feedback, the defibrillator(s) may select one or more optimal vectors that are predicted to be optimal for a defibrillation therapy. In some cases, the defibrillator(s) recommends administration of one or more electrical shocks along the optimal vector(s). For instance, the defibrillator(s) recommends administration of a multi-shock therapy (e.g., a DSD therapy) including multiple electrical shocks along multiple optimal vectors.

Implementations of the present disclosure will now be described with reference to the accompanying figures.

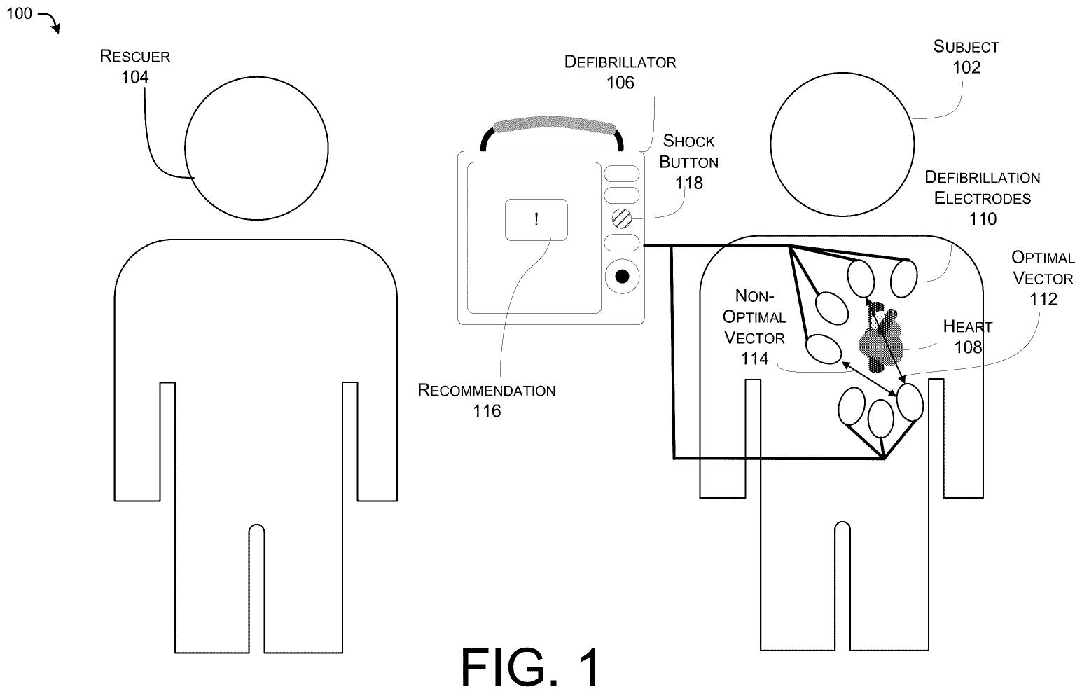

FIG. 1 illustrates an example environment 100 for optimizing defibrillation treatments based on vector selection and vector change. The environment 100, for instance, is a rescue scene in which a subject 102 is experiencing a medical emergency. In various cases, the environment 100 is outside of a clinical setting. For example, the environment 100 is in a public space, such as an airport terminal, school, or office building or a private space, such as a home. The medical emergency of the subject 102 is serious and sudden. For instance, the subject 102 has suddenly and unexpectedly lost consciousness in the environment 100. Accordingly, the subject 102 is provided medical care before being transported to a clinical setting, such as a hospital.

A rescuer 104 is deployed to the environment 100 to provide medical assistance to the subject 102. For example, the rescuer 104 has traveled to and arrived at the environment 100 in response to a call for emergency services from a bystander. In some cases, the rescuer 104 has specific medical training. For example, the rescuer 104 is an emergency medical services (EMS) provider.

In various cases, a rescuer 104 operates a defibrillator 106 to monitor and treat the subject 102. In various cases, the defibrillator 106 is a portable medical device. For example, the defibrillator 106 is transported on an ambulance and carried by the rescuer 104 to the vicinity of the subject 102. The defibrillator 106, for instance, includes one or more on-board power sources (e.g., batteries) configured to provide power to various elements of the defibrillator 106 in the environment 100.

The defibrillator 106 is communicatively coupled with and/or includes one or more sensors configured to detect one or more physiological parameters. Examples of the sensor(s) include one or more electrodes, an ultrasound transducer (e.g., configured to detect physiological structures and/or fluid movement using the Doppler effect), an oximetry sensor, a flow sensor, an oxygen sensor, a carbon dioxide sensor, a blood pressure sensor (e.g., a blood pressure cuff, catheter sensor, etc.), a microphone, an accelerometer, a gyroscope, and the like. Examples of the physiological parameter(s) include electrocardiogram (ECG), electroencephalogram (EEG), blood flow parameters (e.g., instantaneous blood velocity, volumetric blood flow, etc.), pulse rate, heart rate, blood oxygenation (e.g., regional oxygenation, pulse oximetry, oxygen saturation, etc.), airway parameters (e.g., flow rate of air in the airway, a partial pressure of oxygen and/or carbon dioxide in the airway, airway pressure, etc.), blood pressure (e.g., pulse pressure, systolic blood pressure, diastolic blood pressure, mean arterial blood pressure, etc.), physiological sounds (e.g., heart sounds, breath sounds, etc.), and the like. In some cases, the defibrillator 106 is configured to output indications of the physiological parameter(s) to the rescuer 104. The rescuer 104, for instance, identifies a condition of the subject 102 based on the physiological parameter(s). In some examples, the defibrillator 106 automatically analyzes the physiological parameter(s) and outputs a result of the analysis. For instance, the defibrillator 106 outputs a notification that the physiological parameter(s) of the subject 102 are indicative of the condition.

In various cases, the condition of the subject 102 is treatable by an electrotherapy including the administration of one or more electrical shocks to the subject 102. An electrotherapy including administration of a single electrical shock or pulse may be referred to as a “single-shock therapy.” An electrotherapy including administration of multiple electrical shocks or pulses may be referred to as a “sequential-shock therapy” or a “multi-shock therapy.” In some cases, a heart 108 of the subject 102 is exhibiting a shockable arrhythmia. In some examples, the shockable arrhythmia is ventricular fibrillation (VF) or pulseless ventricular tachycardia (VT), which responds to administration of a high-energy defibrillation shock. In various cases, the heart 108 is exhibiting another type of arrhythmia that can respond to electrical stimulation. For example, the heart 108 is exhibiting bradycardia that is treatable by administration of lower-energy pacing pulses. In some examples, the shockable arrhythmia is atrial fibrillation (AF), which is treatable by the administration of one or more electrical shocks synchronized with an R-wave and/or QRS complex indicated in the ECG.

In some examples, the rescuer 104 may apply a single pair of defibrillation electrodes to the chest of the subject 102. Upon identifying that the subject 102 has the condition treatable by the electrical shock(s), the rescuer 104 would cause the defibrillator 106 to administer the electrical shock(s) to the single pair of defibrillation electrodes. However, if the rescuer 104 has misapplied the position of the defibrillation electrodes to the chest of the subject 102, or the subject 102 has some unique physiology that would prevent the rescuer 104 from accurately anticipating the position of the heart 108 within the chest of the subject 102, various problems can occur. For instance, the electrical shock(s) could potentially deliver only a fraction of their energy to the heart of the subject. In some cases, the electrical shock(s) are unable to deliver an expected electrical dosage to the heart 108 of the subject, and therefore are unable to treat the condition of the subject 102. As a result, the condition of the subject 102 may persist, which can cause harm to the subject 102. In certain cases, in which the subject 102 is exhibiting VF, the application of electrical shocks to the single pair of defibrillation electrodes may fail to terminate the arrhythmia, resulting in the subject 102 developing refractory VF.

Various implementations of the present disclosure address these and other problems by testing, selecting, and/or applying electrical stimulation to the subject 102 along multiple vectors. In some examples, multiple defibrillation electrodes 110 are disposed on the chest of the subject 102. For example, more than two defibrillation electrodes 110 are applied to the subject 102. The defibrillation electrodes 110 are electrically connected with the defibrillator 106. For example, the defibrillation electrodes 110 are electrically connected with one or more treatment circuits within the defibrillator 106. In various cases, the defibrillation electrodes 110 are connected to the defibrillator 106 via one or more cables, which are removably connected with one or more ports of the defibrillator 106 via one or more connectors.

In some cases, a single electrode pad includes multiple electrodes among the defibrillation electrodes 110, which can simplify the application of the defibrillation electrodes 110 to the chest, back, torso, or other portion of the subject 102. For example, the rescuer 104 applies the defibrillation electrodes 110 by adhering one or more electrode pads to the chest of the subject 102. In various cases, the defibrillation electrodes 110 are arrayed along the chest of the subject 102. For example, the defibrillation electrodes 110 are disposed along one or more arcs that are disposed on multiple sides of the heart 108 of the subject 102. In some cases, the defibrillation electrodes 110 are all disposed on skin on the chest of the subject 102. In some cases, one or more of the electrodes 110 are disposed on skin on the back of the subject 102. For example, the defibrillation electrodes 110 may be positioned anteriorly and/or posteriorly.

Multiple vectors extend between various pairs of the defibrillation electrodes 110. As used herein, the term “vector,” and its equivalents, refers to a direction and position of an electrical path extending between multiple electrodes. The vectors, for example, include one or more vectors that extend through at least a portion of the heart 108 of the subject 102. In various cases, different vectors extending between the defibrillation electrodes 110 are associated with different efficacies. That is, an electrical shock administered along one vector may successfully treat the condition of the subject 102, whereas an electrical shock administered along a different vector may unsuccessfully treat the condition of the subject 102. Accordingly, it may be beneficial to select a subset of the vectors between the defibrillation electrodes 110 for therapy administration.

In various implementations, the defibrillator 106 is configured to select, among the vectors, an optimal vector 112 for administering an electrical therapy to the heart 108 of the subject 102. In various cases, energy from an electrical signal output along the optimal vector 112 is predicted to be significantly delivered to the heart 108 of the subject 102. For instance, the optimal vector 112 may extend through at least a portion of the heart 108. One or more techniques can be utilized to identify the optimal vector 112 among the multiple vectors extending between the defibrillation electrodes 110.

In some examples, the defibrillator 106 selects the optimal vector 112 by analyzing feedback from one or more test shocks administered to the defibrillation electrodes 110. As used herein, the term “test shock,” and its equivalents, refers to an electrical pulse with a defibrillation energy level or a sub-defibrillation energy level. A test shock, in some cases, has a similar shape and/or duration to that of a defibrillation shock. For example, a test shock can be multiphasic (e.g., biphasic). In various examples, a test shock has an energy level in a range of 0.1 J to 200 J, such as a range of 0.1 J to 5 J. In some examples, a test shock is a defibrillating electrical shock. For example, a test shock can have an energy level in a range of 200 J to 360 J.

In some cases, the defibrillator 106 monitors one or more physiological parameters of the subject 102 in response to administration of a test shock along the optimal vector 112. For example, the defibrillator 106 identifies the optimal vector 112 by detecting a perturbance in the ECG of the subject 102 in response to the application of the test shock. As used herein, the term “perturbance,” and its equivalents, can refer to a temporary or permanent change. In some cases, the perturbance is a different heart rhythm than the shockable heart rhythm that was present before the test shock. For example, if the ECG indicates that the heart 108 is in VF prior to the test shock but indicates a non-VF heart rhythm for at least a predetermined time period (e.g., 10 ms to 5 seconds, or 1 to 5 seconds after application of the test shock), then the defibrillator 106 may detect the temporary transition as a perturbance of the VF. The perturbance, for instance, includes one or more of a change in VF rate (e.g., a transient decrease in VF rate), a change in VF amplitude (e.g., a transient increase in peaks within the ECG indicative of VF), a change in AMSA of the ECG, or any combination thereof. In various cases, a perturbance includes one or more temporary changes in the ECG. In various cases, if a test shock causes a perturbance when applied to a pair of the defibrillation electrodes 110, then the defibrillator 106 infers that the vector extending between the pair of the defibrillation electrodes 110 is the optimal vector 112. In contrast, the defibrillator 106 may refrain from detecting a perturbance if the VF continues through the application of the test shock. For instance, if a test shock does not cause a perturbance when applied to a pair of the defibrillation electrodes 110, then the defibrillator 106 infers that the vector extending between the pair of the defibrillation electrodes 110 is a non-optimal vector 114. The test shocks, for example, enable the defibrillator 106 to infer one or more electrical paths that are optimal for terminating a shockable rhythm of the heart 108. The existence or absence of the perturbance, for instance, is a type of feedback.

In some examples, the defibrillator 106 identifies the optimal vector 112 and the non-optimal vector 114 by observing a response to the administration of an electrotherapy across the optimal vector 112 and the non-optimal vector 114. For example, the defibrillator 106 may administer a first electrotherapy (e.g., an electrical shock) across the non-optimal vector 114, wherein the first electrotherapy is configured to treat a medical condition (e.g., VF). The defibrillator 106, in various cases, analyzes one or more physiological parameters (e.g., ECG) in order to determine whether the medical condition was at least temporarily resolved after the administration of the first electrotherapy. The defibrillator 106 may identify the non-optimal vector 114 by determining that the medical condition is continuous after the administration of the first electrotherapy. In response to identifying the non-optimal vector 114, the defibrillator 106 may switch vectors. For instance, the defibrillator 106 outputs a second electrotherapy (e.g., another electrical shock) across the optimal vector 112. The defibrillator 106 may, for instance, identify the optimal vector 112 by determining that the condition is at least temporarily resolved by the administration of the second electrotherapy.

Some implementations of the present disclosure enable the selection of the optimal vector 112 for the purposes of administering a pacing therapy. In various cases, the defibrillator 106 identifies the optimal vector 112 by determining that pacing pulses output along the optimal vector 112 result in electrical capture of the heart 108. In particular examples, the defibrillator 106 detects that the subject 102 has bradycardia. In response, the defibrillator 106 outputs first pacing pulses to electrodes associated with a first vector (e.g., the non-optimal vector). In response to administering the first pacing pulses, the defibrillator 106 analyzes one or more physiological parameters of the subject 102 in order to determine whether the first pacing pulses successfully resulted in electrical capture of the heart 108. In some cases, the defibrillator 106 inputs one or more of an ECG feature (e.g., a T-wave or QRS complex), a blood oxygenation, a plethysmographic waveform (e.g., sampled from a pulse oximeter), a magnitude of oscillation in a detected plethysmographic waveform, a blood pressure, or an EtCO₂ of the subject 102 into a computing model configured to calculate a likelihood that capture has been achieved. Techniques for automatically identifying pacing capture are described, for instance, in US Pub. No. 2022/0219000, which is incorporated by reference herein in its entirety. In some cases, the defibrillator 106 determines that the first vector is the non-optimal vector 114 by determining that the first pacing pulses do not result in electrical capture of the heart 108. In response to determining that the first vector is the non-optimal vector 114, the defibrillator 106 may administer second pacing pulses to a second vector that is different than the first vector. For example, the defibrillator 106 may determine that the second vector is the optimal vector 112 in response to determining that the second pacing pulses result in electrical capture of the heart 108.

In various cases, once the defibrillator 106 identifies the optimal vector 112 based on a first type of electrotherapy, the defibrillator 106 is configured to apply a second type of electrotherapy across the same optimal vector 112. For example, if the defibrillator 106 identifies the optimal vector 112 by detecting electrical capture in response to pacing pulses being applied to the subject 102 across the optimal vector 112, the defibrillator 106 may select the optimal vector 112 for the application of an electrical shock if the subject 102 subsequently exhibits VF.

In some cases, the defibrillator 106 selects the optimal vector 112 by analyzing ECG leads of the subject 102 measured along the multiple vectors. An ECG lead, for instance, is detected by detecting an electrical potential (voltage) between a pair of electrodes in the defibrillation electrodes 110. In various cases, at least a portion of the electrical potential is output by the heart of the subject 102. Accordingly, the electrical potential is indicative to the electrical functionality of the heart.

The defibrillator 106, for instance, samples an ECG lead between a pair of the defibrillation electrodes 110 over an extended time period. In various cases, the ECG lead is sampled during a time period in which the subject 102 is not receiving chest compressions. Alternatively, a chest compression artifact is removed from the ECG lead, if the ECG lead is sampled when the subject 102 is receiving chest compressions.

During the time period, the heart 108 of the subject 102 may be spontaneously outputting an electrical signal that is detected by each ECG lead. Different ECG leads captured from the same subject 102 have different waveforms, in various cases. The differences between the ECG leads relate to the geometric relationship between the electrodes from which the ECG leads are detected and/or the position of the heart 108 of the subject 102. In particular cases, amplitudes and/or frequencies of different ECG leads reflect the presence (or an amount) of the heart 108 that is within an electrical path extending between the electrodes detecting the different ECG leads. Thus, the defibrillator 106 may infer that the heart 108 is located along a vector between a pair of defibrillation electrodes 110 by detecting greater than a threshold amplitude and/or frequency of the ECG lead detected between the pair of the defibrillation electrodes 110. In some cases, the defibrillator 106 compares a peak amplitude, an average peak amplitude, or an average amplitude to at least one threshold. According to some implementations, if the amplitude of an ECG lead is greater than the at least one threshold, the defibrillator 106 determines that the associated vector is the optimal vector 112. In various examples, if the amplitude of an ECG lead is less than the at least one threshold, the defibrillator 106 determines that the associated vector is the non-optimal vector 114. In various implementations, the defibrillator 106 identifies the optimal vector 112 based on the ECG lead that has the highest amplitude among the ECG leads detected by the defibrillator 106.

In some examples, the defibrillator 106 compares a frequency of VF oscillations in the ECG leads to at least one threshold. If the frequency of an ECG lead is greater than the at least one threshold, the defibrillator 106 determines that the associated vector is the optimal vector 112, for instance. In various cases, if the frequency of an ECG lead is less than the at least one threshold, the defibrillator 106 determines that the associated vector is the non-optimal vector 114. In various cases, the defibrillator 106 identifies the optimal vector 112 based on the ECG lead that has the highest frequency among the ECG leads detected by the defibrillator 106.

According to some implementations, the defibrillator 106 identifies the optimal vector 112 and the non-optimal vector 114 based on a variance of amplitude and/or frequency in each ECG lead. In some implementations, termination of a rotor of the heart of the subject 102 can cause resolution of the VF. In various examples, if an ECG lead goes through a phase singularity of the reentrant rhythm, the ECG lead may exhibit multiple transitions between patterns of high-amplitude-and-low-frequency rhythms and low-amplitude-and-high-frequency rhythms due to the presence of a rotor in the electrical activity of the heart. This pattern, for instance, indicates that the corresponding vector corresponds to the center of the rotor. In various cases, the defibrillator 106 infers that an ECG lead corresponds to the optimal vector 112 upon determining that the ECG lead exhibits VF that transitions between greater than a threshold amplitude and less than a threshold frequency to less than a threshold amplitude and greater than a threshold frequency.

In some cases, the defibrillator 106 is further configured to identify the optimal vector 112 and/or the non-optimal vector 114 by comparing transthoracic impedances associated with the vectors between the defibrillation electrodes 110. A transthoracic impedance, for instance, is detected by applying an electrical signal across a pair of the defibrillation electrodes 110, detecting an electrical signal resulting from the applied electrical signal, and determining the impedance based on the applied and detected electrical signals. For example, various parts of the heart 108 (e.g., atria and ventricles) expand and/or contract during the time period in which the transthoracic impedance is detected. If one or more portions of the heart 108 are located along the electrical path between the pair of defibrillation electrodes 110, then the transthoracic impedance signal will change over time in accordance with the movement of the heart 108. Thus, the optimal vector 112 and/or the non-optimal vector 114, in some cases, are identified based on variances in the transthoracic impedances along the different vectors.

In some cases, an analysis of transthoracic impedances can enable the defibrillator 106 to select between multiple adequate vectors. For example, if the defibrillator 106 determines that the peak amplitudes of two ECG leads, associated with a first vector and a second vector, are above at least one threshold, the defibrillator 106 may select the first vector as the optimal vector 112 by determining that a transthoracic impedance associated with the first vector is lower than a transthoracic impedance associate with the second vector. For instance, the lower transthoracic impedance associated with the first vector may indicate that the electrical path between the pair of defibrillation electrodes 110 associated with the first vector is more targeted toward the heart 108 than the electrical path between the pair of defibrillation electrodes 110 associated with the second vector.

The defibrillator 106 is configured to recommend and/or cause a treatment to be applied across the optimal vector 112. In some examples, the defibrillator 106 refrains from applying the treatment across the non-optimal vector 114. For instance, upon identifying the optimal vector 114, the defibrillator 106 may recommend, or apply, at least one electrical shock to the defibrillation electrodes 110 associated with the optimal vector 112. In various cases, the selection of the optimal vector 112 enhances the likelihood that the treatment will be successful. For instance, an electrical shock applied to the optimal vector 112 may have a greater likelihood of resolving VF of the heart 108 than an electrical shock applied to the non-optimal vector 114.

In particular cases, the defibrillator 106 outputs a recommendation 116 to the rescuer 104 based on the optimal vector 112. In some examples, the recommendation 116 is output on a display of the defibrillator 106. In some cases, the recommendation 116 is output via an audible alert, haptic feedback, or some other mechanism for communication to the rescuer 104. The recommendation 116 indicates at least one of the optimal vector 112, the defibrillation electrodes 110 associated with the optimal vector 112, or a recommendation to administer a therapy to the defibrillation electrodes 110. In some cases, the recommendation 116 instructs the rescuer 104 to administer an electrical shock across the optimal vector 112.

According to some implementations, the defibrillator 106 outputs the therapy across the optimal vector 112. For example, the defibrillator 106 outputs one or more electrical signals (e.g., an electrical shock, pace pulses, etc.) to the optimal vector 112, thereby treating the condition of the subject 102. In various cases, the defibrillator 106 administers the therapy in response to receiving an input signal from the rescuer 104. For instance, the defibrillator 106 may output the therapy in response to detecting that the rescuer 104 has pressed a shock button 118 of the defibrillator. In some examples, the therapy includes an electrical shock that has a higher energy than the test shocks.

Although FIG. 1 illustrates a single optimal vector 112 and a single non-optimal vector 114, implementations are not so limited. For example, in some cases, multiple optimal vectors and/or multiple non-optimal vectors are identified by the defibrillator 106. In some implementations, the defibrillator 106 outputs respective therapies to respective combinations of the defibrillation electrodes 110 corresponding to the multiple optimal vectors. For instance, the defibrillator 106 outputs a first electrical shock to a first optimal vector, and subsequently outputs a second electrical shock to a second optimal vector if the subject 102 refibrillates after administration of the first electrical shock. That is, in some cases, the defibrillator 106 rotates therapies among multiple optimal vectors associated with various combinations of the defibrillation electrodes 110. According to some cases, the change in vectors may enhance the likelihood that the condition of the heart 108 of the subject 102 will be resolved.

In some cases, the defibrillator 106 administers, at least in part, a multi-shock (e.g., double-sequential defibrillation (DSD)) therapy along the optimal vector 112. The rescuer 104 and/or the defibrillator 106 may determine that the subject 102 has a condition that warrants the multi-shock therapy. For instance, the defibrillator 106 may detect that the subject 102 has VF that has not responded to one or more previous defibrillation shocks administered to the subject 102 (e.g., refractory VF). In response to detecting the condition that warrants the multi-shock therapy, the defibrillator 106 may administer to the subject 102, multiple sequential electrical shocks along the optimal vector 112 or multiple optimal vectors. For example, the defibrillator 106 may administer a first electrical shock along the optimal vector 112, a second electrical shock along another optimal vector, wherein the first electrical shock and the second electrical shock temporally overlap. In some cases, the defibrillator 106 includes multiple therapy circuits, each with a respective capacitor, such that the defibrillator 106 may output the multiple electrical shocks along the different vectors. For instance, the defibrillator 106 outputs multiple electrical shocks along different paths through different combinations of the defibrillation electrodes 110.

In some cases, the multi-shock therapy is coordinated between the defibrillator 106 and a separate defibrillator (not illustrated) applied to the subject 102. In some cases, the separate defibrillator is an implantable defibrillator implanted inside of the body of the subject 102. In some examples, the separate defibrillator is a wearable defibrillator that is worn by the subject 102. In some cases, the separate defibrillator is an external defibrillator, such as a separate monitor-defibrillator or AED. The separate defibrillator, in some cases, is electrically connected to the defibrillation electrodes 110, such that the defibrillator 106 outputs the first electrical shock along the optimal vector 112 and the separate defibrillator outputs the second electrical shock along a different vector between the defibrillation electrodes 110. In some examples, the separate defibrillator is connected to separate defibrillation electrodes (not illustrated) that are disposed on the body of the subject 102. For instance, the separate defibrillator outputs the electrical shock along another vector between the separate defibrillation electrodes. In various cases, the rescuer 104 manually activates both defibrillators, the defibrillator 106 outputs a communication signal to the separate defibrillator that coordinates the timing of the electrical shocks, the separate defibrillator outputs a communication signal to the defibrillator 106 that coordinates the timing of the electrical shocks, or an additional device outputs communication signals to the defibrillator 106 and the separate defibrillator that coordinates the timing of the electrical shocks. In some examples, the multi-shock therapy may effectively treat the condition of the subject 102 in circumstances in which a single electrical shock is unable to treat the condition.

Although not illustrated in FIG. 1, any of the vectors described herein can include virtual vectors. As used herein, the term “virtual vector,” and its equivalents, can refer to a vector that results from a combination of more than two electrodes. For example, a virtual vector may extend from a first electrode among the defibrillation electrodes 110 to second and third electrodes among the defibrillation electrode 110, such that the virtual vector extends along a position between the second and third electrodes. A test shock, an electrical therapy, or other type of electrical signal can be applied to the virtual vector by activating the first, second, and third electrodes, for instance.

A specific example will now be described with reference to FIG. 1. The subject 102, for instance, has collapsed in the environment 100. In response, the rescuer 104 applies one or more defibrillation pads that include the defibrillation electrodes 110 to the skin of the chest of the subject 102. The defibrillator 106 detects the ECG of the subject 102 and detects the VF by analyzing the ECG. Upon initially detecting the VF, the defibrillator 106 outputs the recommendation 116 indicating an instruction to administer a first electrical shock to the subject 102. The rescuer 104 presses the shock button 118 and the defibrillator 106 outputs the first electrical shock at an energy level of 360 J to one of the vectors extending between the defibrillation electrodes 110.

The defibrillator 106 further determines, by analyzing the ECG, that the VF of the subject 102 did not even temporarily resolve after administration of the first electrical shock. The defibrillator 106 infers that the heart 108 of the subject has a shock-resistant arrhythmia, such as refractory VF. Thus, the defibrillator 106 may infer that a subsequent therapy should be modified to enhance the likelihood of resolving the VF. In some examples, the defibrillator 106 once again outputs the recommendation 116 to administer an electrical shock to the subject. Further, the rescuer 104 presses the shock button 118 a second time.

In response to detecting the press of the shock button 118 by the rescuer 104, the defibrillator 106 causes administration of one or more second electrical shocks to the subject 102. In some cases, the defibrillator 106 automatically outputs a single second electrical shock along a different vector than the one applied by the first electrical shock. For instance, if the first electrical shock was along the non-optimal vector 114, the second electrical shock may be along the optimal vector 112. In some implementations, the defibrillator 106 analyzes feedback obtained before the rescuer 104 presses the shock button 118 the second time to specifically identify the optimal vector 112. For example, the defibrillator 106 may utilize ECG leads detected between the defibrillation electrodes 110 to identify the optimal vector 112. In some examples, the defibrillator 106 administers small test shocks (e.g., at an energy level of 20 J each) to various vectors defined by the defibrillation electrodes 110 and identifies the optimal vector 112 based on a test shock that caused a perturbance in the VF of the subject 102.

In some cases, the defibrillator 106 causes administration of a multi-shock therapy to the subject 102. For instance, the defibrillator 106 may output multiple second electrical shocks to multiple vectors between the defibrillation electrodes 110, including the optimal vector 112. In some cases, the defibrillator 106 causes an additional defibrillator to output one of the second electrical shocks in the multi-shock therapy. In various cases, the VF of the subject 102 may be resolved in response to the delivery of the second electrical shock(s).

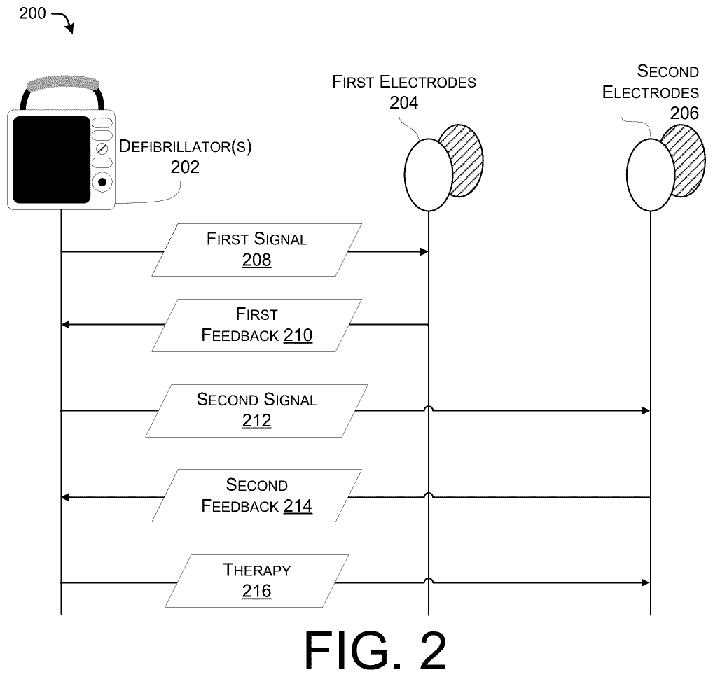

FIG. 2 illustrates example signaling 200 for selecting an optimal vector for administering a therapy to a subject. The signaling 200, for instance, is between one or more defibrillators 202, first electrodes 204, and second electrodes 206. In some cases, the defibrillator(s) 202 include the defibrillator 106. In particular instances, the defibrillator(s) 202 include multiple defibrillators. In some examples, the defibrillation electrodes 110 include the first electrodes 204 and/or the second electrodes 206. The first electrodes 204 and the second electrodes 206 are applied to a subject, for instance. A first vector extends between the first electrodes 204. A second vector extends between the second electrodes 206. The first vector and the second vector include at least one virtual vector, in some cases.

The defibrillator(s) 202 transmit a first signal 208 to the first electrodes 204. The first signal 208 is an electrical signal. For instance, the first signal 208 is a test shock, an electrical signal for detecting an ECG lead and/or transthoracic impedance between the first electrodes 204, a pace pulse, or an electrotherapy, such as an electrical shock suitable for defibrillation or synchronized cardioversion, pacing pulses, or the like.

The first electrodes 204 return first feedback 210 to the defibrillator(s) 202. In some cases, the first feedback 210 includes the ECG lead of a subject along the first vector. For example, the defibrillator(s) 202 determine, based on the feedback 210, that the first signal 208 has not caused a perturbance in the ECG and/or a shockable arrhythmia indicated by the ECG. In some implementations, the first feedback 210 includes, or is indicative of, the ECG lead itself between the first electrodes 204. In some examples, the defibrillator(s) 202 determine, by determining that an amplitude (e.g., a peak amplitude) of the ECG lead is below a threshold, that a therapy applied to an electrical path between the first electrodes 204 is not predicted to resolve the condition of the subject. In various examples, the defibrillator(s) 202 determine that the first vector is a non-optimal vector based on the first feedback 210.

Further, the defibrillator(s) 202 output a second signal 212 to the second electrodes 206. The second signal 212 is an electrical signal. For instance, the second signal 212 is a test shock, an electrical signal for detecting an ECG lead and/or transthoracic impedance between the second electrodes 206, a pacing pulse, or an electrical shock suitable for defibrillation or synchronized cardioversion.

The second electrodes 206 return second feedback 214 to the defibrillator(s) 202. In some cases, the second feedback 214 includes the ECG of a subject along the second vector. For example, the defibrillator(s) 202 determine, based on the second feedback 214, that the second signal 212 has caused a perturbance in the ECG and/or a shockable arrhythmia indicated by the ECG. In some implementations, the second feedback 214 includes, or is indicative of, an ECG lead between the second electrodes 206. In some examples, the defibrillator(s) 202 determine, by determining that a peak amplitude of the ECG lead is above a threshold, that a therapy applied to an electrical path between the second electrodes 206 is predicted to resolve the condition of the subject. In various cases, the defibrillator(s) 202 determine that the second vector is an optimal vector based on the second feedback 214.

In response to receiving the second feedback 214, the defibrillator(s) 202 output a therapy 216 to the second electrodes 206. The therapy 216, for instance, includes one or more electrical signals configured to treat the condition of the subject. In some cases, the therapy 216 includes an electrical shock suitable for defibrillation and/or synchronized cardioversion. In some examples, the therapy 216 includes pace pulses. In various implementations, the therapy 216 includes a first electrical shock included in a DSD therapy, which also includes a second electrical shock administered to the subject by the defibrillator(s) 202. The first electrical shock and the second electrical shock, for instance, are temporally overlapping.

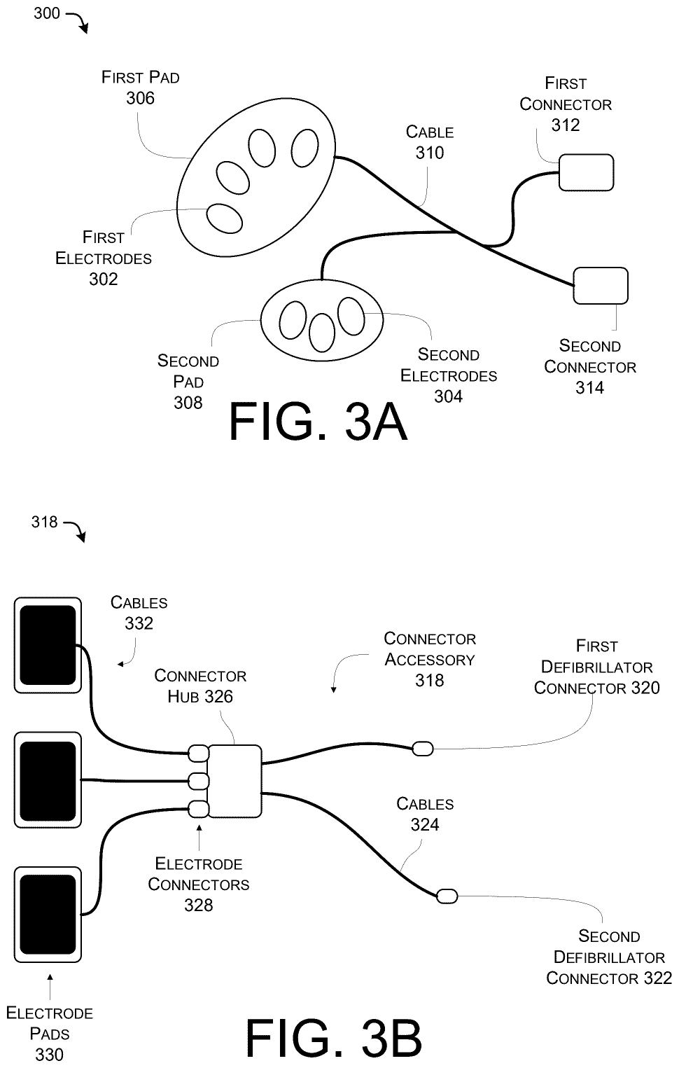

FIG. 3A illustrates an example electrode accessory 300 suitable for automated vector change and selection. The electrode accessory 300 can be referred to as an electrode assembly. For example, the electrode accessory 300 may be removably connected to the defibrillator 106. The electrode accessory 300 includes various defibrillation electrodes including first electrodes 302 and second electrodes 304. In various cases, the first electrodes 302 and the second electrodes 304 include the defibrillation electrodes 110, the first electrodes 204, the second electrodes 206, or any combination thereof. The first electrodes 302 and the second electrodes 304, in various cases, include an electrically conductive material. For example, the first electrodes 302 and the second electrodes 304 include silver/silver chloride (Ag/AgCl) electrodes, nickel, or any other material suitable for defibrillation electrodes. In some cases, the first electrodes 302 and the second electrodes 304 are coated with an electrically conductive gel, such as a hydrogel including one or more electrolytes.

The first electrodes 302 are disposed on a first pad 306. The second electrodes 304 are disposed on a second pad 308. In various implementations, the first pad 306 and the second pad 308 are electrically insulative substrates. For instance, the first pad 306 and the second pad 308 include a polymer foam. In some cases, at least a portion of the first pad 306 and the second pad 306 is coated with a biocompatible adhesive configured to adhere the first pad 306 and the second pad 308 to skin of a subject. In various implementations, the first electrodes 302 are disposed on the first pad 306 along a first arc. The second electrodes 304 are disposed on the second pad 308 along a second arc, for instance.

A cable 310 physically couples the first pad 306 and the second pad 308 to a first connector 312 and a second connector 314. In various cases, the cable 310 includes one or more conductive wires that are electrically coupled with the first electrodes 302 and the second electrodes 304. The conductive wires, in various cases, are also electrically coupled with the first connector 312 and the second connector 314. The first connector 312 is configured to be removably coupled with a port of a defibrillator. In various cases, the second connector 314 is configured to be removably coupled with another port of the defibrillator, or a port of a separate defibrillator. The cable 310, in various cases, further includes an electrically insulative coating or covering that shields the wires from each other, and from external signals and interference.

In various cases, the cable 310 houses several electrically isolated paths between combinations of the first electrodes 302, the second electrodes 304, the first connector 312, and the second connector 314. For instance, a first path may extend from a first electrode among the first electrodes 302, a second path may extend from a second electrode among the first electrodes 302, a third path may extend from a first electrode among the second electrodes 304, and a fourth path may extend from a second electrode among the second electrodes 304. In some cases, the first path and the third path are connected with the first connector 312, whereas the second path and the fourth path are connected with the second connector 314. In some cases, one or more switches within the cable 310 can selectively connect one or more of the paths to the first connector 312, the second connector 314, or both. In some examples, by operation of the switches, only one of the first connector 312 or the second connector 314 is connected with a given path at a single time. According to some examples, more than two paths are connected to the first connector 312 and more than two paths are connected to the second connector 314.

In various cases, the defibrillator(s) detect one or more ECG leads via the paths within the accessory 300. Further, in some cases, the defibrillator(s) output one or more signals along multiple vectors defined between the first electrodes 302 and the second electrodes 304 along the paths within the cable 310. These signals, for instance, may include test shocks, electrical shocks, pacing pulses, and other electrical signals described herein. Thus, the accessory 300 may enable analysis of multiple vectors between the first electrodes 302 and the second electrodes 304 via various techniques described herein.

In some examples, the accessory 300 facilitates the administration of a multi-shock therapy in which one or more defibrillators output multiple electrical shocks to a subject along multiple vectors. For example, a first defibrillator coupled with the first connector 312 may output a first electrical shock to one of the first electrodes 302 and one of the second electrodes 304 via first paths through the cable 310, and a second defibrillator coupled with the second connector 314 may output a second electrical shock to one of the first electrodes 302 and one of the second electrodes 304 via second paths through the cable 310. In some cases, the first defibrillator is a monitor-defibrillator and the second defibrillator is an AED, for instance. The first paths and the second paths in various cases, are electrically shielded from one another by the cable 310.

FIG. 3B illustrates an example environment 316 including a connector accessory 318 configured to facilitate administration of electrotherapies at different vectors. The connector accessory 318 includes a first defibrillator connector 320 configured to be removably coupled with a first defibrillator. In various cases, the connector accessory 318 further includes a second defibrillator connector 322 configured to be removably coupled with a second defibrillator. In various cases, the first defibrillator connector 320 is configured to be physically and electrically coupled with a port of the first defibrillator, and the second defibrillator connector 322 is configured to be physically and electrically coupled with a port of the second defibrillator. For instance, the first defibrillator connector 320 and the second defibrillator connector 322 are plugs configured to be coupled with output ports of the first and second defibrillators, respectively.

The connector accessory 318 further includes cables 324 that extend between the first defibrillator connector 320 and a connector hub 326, as well as between the second defibrillator connector 322 and the connector hub 326. In various cases, the connector hub 326 includes a housing that includes ports that are configured to be physically and electrically coupled with multiple electrode connectors 328. For example, the electrode connectors 328 are configured to be removably coupled to the ports of the connector hub 326. In various cases, the electrode connectors 328 are electrically coupled to various electrode pads 330. For instance, respective cables 332 connect the electrode connectors 328 to the electrode pads 330. In some cases, the electrode connectors 328 are physically and electrically equivalent to the first defibrillator connector 320 and the second defibrillator connector 322. For example, in some cases, the electrode connectors 328 are configured to be removably connected with the first defibrillator and the second defibrillator. In various cases, the electrode pads 330 are configured to be disposed on skin of a subject. For example, when placed on the subject, pairs of the electrode pads 330 define multiple vectors that intersect the heart of the subject along different paths and angles.

In various implementations, the connector accessory 318 is configured to output one or more electrotherapies output by the first defibrillator and/or the second defibrillator along one or more of the vectors defined by the electrode pads 330. In particular cases, the connector accessory 318 includes a circuit that selectively outputs one or more electrical signals by the first defibrillator and/or the second defibrillator provided to the first defibrillator connector 320 and/or the second defibrillator connector 322 along the different vectors. For example, the circuit includes one or more switches (e.g., transistors) configured to selectively electrically connect different pairs of the electrode pads 330 to the first defibrillator connector 320 and/or the second defibrillator connector 322. The switch(es), in some cases, are configured to selectively disconnect one or more of the electrode pads 330 from the first defibrillator connector 320 and/or the second defibrillator connector 322. The circuit, in some cases, is housed within the connector hub 326.

In particular cases, the connector accessory 318 is configured to change vectors of electrotherapies provided to the first defibrillator connector 320 by the first defibrillator and/or provided to the second defibrillator connector 322 by the second defibrillator. For example, the connector accessory 318 is configured to control the switch(es) to cycle consecutive electrotherapies (e.g., electrical shocks, pacing pulses, etc.) along different vectors of the electrode pads 330. In some cases, the connector hub 326 is configured to identify, or receive an instruction identifying, an optimal vector. For example, the connector hub 326 may selectively connect the first and/or second defibrillator to the optimal vector by operating the switch(es).

In some cases, the connector accessory 318 is configured to apply electrotherapies output by the first defibrillator and the second defibrillator as a multi-shock therapy (e.g., a DSD therapy or other sequential-shock therapy). In some examples, the connector accessory 318 receives a first electrical shock from the first defibrillator via the first defibrillator connector 320 and a second electrical shock from the second defibrillator via the second defibrillator connector 322. In particular cases, the connector accessory 318 ensures that the first electrical shock and the second electrical shock are delivered to one or more vectors of the electrode pads 330 at a precise timing that can enhance the efficacy of the multi-shock therapy.

In addition to routing the first electrical shock and the second electrical shock to appropriate vectors using the circuit, the connector accessory 318, in some cases, temporarily stores the first electrical shock or the second electrical shock to ensure that both shocks are appropriately delivered at predetermined time intervals. In some examples, the circuit includes one or more capacitors configured to temporarily store the first electrical shock or the second electrical shock, and to discharge the stored electrical shock at a time that corresponds to the discharge of the other electrical shock to the electrode pads 330. For instance, if the first defibrillator connector 320 receives the first electrical shock ten seconds prior to the second defibrillator connector 322 receiving the second electrical shock, a capacitor in the connector hub 326 may temporarily store the first electrical shock. In some cases, the connector hub 326 is configured to detect the second electrical shock received by the second defibrillator connector 322 and to discharge the second electrical shock to the electrode pads 330. Further, the capacitor in the connector hub 326 is configured to discharge the first electrical shock to the electrode pads 330 at a time that is temporally overlapping with the discharge of the second electrical shock. In some cases, the connector accessory 318 enables the administration of the sequential-shock therapy by two defibrillators that are not otherwise specifically designed or configured to administer the sequential-shock therapy.

In some cases, the connector accessory 318 enables a single defibrillator to administer a sequential-shock therapy. For example, the capacitor in the connector accessory 318 may be configured to temporarily store a first electrical shock output by the first defibrillator to the first defibrillator connector 320. Subsequently, the first defibrillator connector 320 may recharge its own capacitor (e.g., using a battery) and later output a second electrical shock to the first defibrillator connector 320. When the second electrical shock is received by the connector accessory 318, in various cases, the circuit is configured to discharge the first electrical shock stored by the capacitor of the connector accessory 318 to the electrode pads 330, and to route the second electrical shock to the electrode pads 330, at a predetermined timing relationship. Accordingly, in some cases, the connector accessory 318 enables the first defibrillator to output multiple sequential electrical shocks at the predetermined timing relationship, even if the defibrillator may have only a single capacitor for outputting electrical shocks.

In some examples, the connector accessory 318 communicates with the first and second defibrillators in order to achieve a sequential-shock therapy. For example, a processor within the connector hub 326 is configured to output a first communication signal to the first defibrillator connector 320 and/or a second communication signal to the second defibrillator connector 322. In response to receiving the first communication signal, the first defibrillator may output a first electrical shock at a first predetermined time interval. In some cases, in response to receiving the second communication signal, the second defibrillator outputs a second electrical shock at a second predetermined time interval. The first predetermined time interval and/or the second predetermined time interval may be selected, by the connector accessory 318, to maximize the therapeutic efficacy of a sequential-shock therapy. Accordingly, the connector accessory 318 can facilitate the timing of the sequential-shock therapy without temporarily storing the first electrical shock or the second electrical shock.

In some cases, the connector accessory 318 includes one or more input devices (e.g., user operational switches) configured to engage one or more of the vectors defined by the electrode pads 330 and/or the capacitor in the circuit of the connector accessory 318. Accordingly, a user can selectively and manually direct the connector accessory 318 to output one or more electrotherapies to predetermined vectors defined by the electrode pads 330 and/or to facilitate the administration of a sequential-shock therapy, as described herein. For instance, the connector accessory 318 is configured to operate via one or more modes selected by the user.

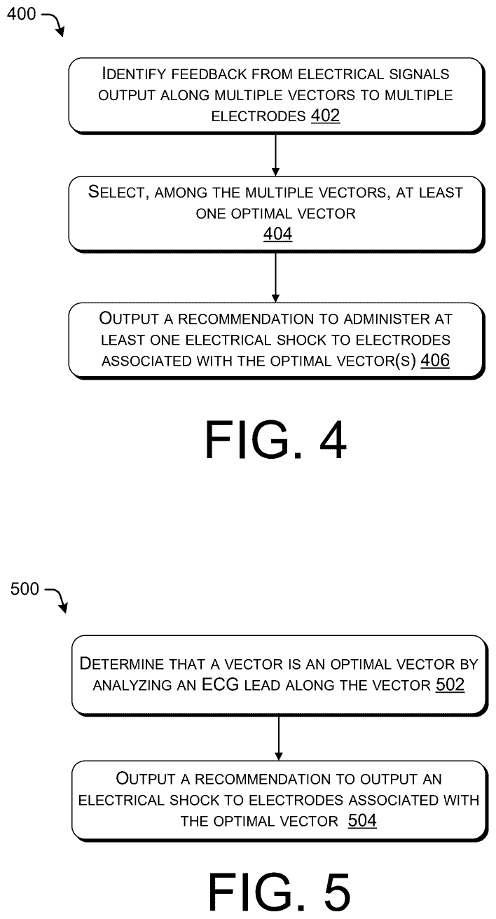

FIG. 4 illustrates an example process 400 for selecting an optimal vector for administration of a therapy to a subject. The process 400 is performed by an entity, which may include at least one of a defibrillator (e.g., the defibrillator 106, the defibrillator(s) 202, etc.), a medical device, a computing device, at least one processor, or any combination thereof.

At 402, the entity identifies feedback from electrical signals output along multiple vectors to multiple electrodes. In some cases, the electrical signals include test shocks. Each of the test shocks, for instance, has a lower energy level than an electrical shock suitable for defibrillation. According to some cases, the electrical signals include previous applications of an electrotherapy, such as pacing pulses or electrical shocks suitable for defibrillation or synchronized cardioversion. In some cases, the feedback includes one or more physiological parameters indicative of an efficacy of the electrotherapy, such as physiological parameter(s) indicative of electrical capture and/or persistence of an arrhythmia (e.g., an ECG indicating whether VF has been temporarily resolved. In some cases, the feedback includes signals indicative of ECG leads and/or transthoracic impedances along the multiple vectors.

At 404, the entity selects, among the multiple vectors, at least one optimal vector. The entity selects the optimal vector(s) based on the feedback. In some cases, the entity selects the optimal vector(s) in response to detecting a perturbation in a shockable rhythm indicated by one or more ECG leads of the subject in response to one or more of the electrical signals (e.g., test shocks, electrotherapy, etc.) being administered to the optimal vector(s). In some cases, the entity selects the optimal vector(s) in response to predicting that greater than a threshold amount of energy from at least one of the electrical shocks output along the optimal vector(s) would be delivered to the heart of the subject. In some cases, the entity selects the optimal vector(s) in response to detecting that an amplitude of a signal in the feedback (e.g., a peak amplitude of an ECG lead during a sampling period) along the optimal vector(s) is greater than a threshold. In some cases, the vectors include at least one virtual vector. The vectors, in various cases, extend between pairs of electrodes that are positioned externally on the subject. In some cases, a single electrode is shared between at least two of the pairs of electrodes.

At 406, the entity outputs a recommendation to administer at least one electrical shock to electrodes associated with the optimal vector(s). In some cases, the entity outputs a recommendation to administer an electrical shock to a single pair of electrodes associated with a single optimal vector. In some examples, the entity outputs a recommendation to administer multiple electrical shocks to multiple pairs of electrodes associated with multiple optimal vectors. For instance, the entity may output a recommendation to administer a DSD therapy including two electrical shocks along two optimal vectors. For instance, the two electrical shocks are temporally overlapping. In some cases, the entity outputs at least one of the electrical shocks to the subject along the optimal vector(s). Other multi-shock therapies (e.g., therapies including more than one electrotherapy administered to the electrodes, sequential-shock therapies including multiple shocks that are administered sequentially, therapies including more than two shocks, etc.) can also be administered along the optimal vector(s).

FIG. 5 illustrates an example process 500 for selecting an optimal vector based on transthoracic impedance. The process 500 is performed by an entity, which may include at least one of a defibrillator (e.g., the defibrillator 106, the defibrillator(s) 202, etc.), a medical device, a computing device, at least one processor, or any combination thereof.

At 502, the entity determines that a vector is an optimal vector by analyzing an ECG lead along the vector. For example, the entity detects the ECG lead along the vector. In some cases, the entity detects the optimal vector by determining that an amplitude of the ECG lead (e.g., an average amplitude during a sampling period, an average peak amplitude during the sampling period, a peak amplitude during the sampling period, etc.) is greater than a threshold. In some cases, the entity determines that another vector is a non-optimal vector by analyzing an ECG lead along the non-optimal vector. For example, the entity may determine that an amplitude of the ECG lead along the non-optimal vector is less than a threshold. In some cases, the entity determines that a transthoracic impedance detected along the optimal vector is less than a threshold, or is less than a transthoracic impedance detected along the non-optimal vector.

At 504, the entity outputs a recommendation to administer an electrical shock to electrodes associated with the optimal vector. In some cases, the entity outputs the recommendation in response to determining that at least one ECG lead of the subject is indicative of VF or pulseless VT. In some cases, the entity detects the ECG lead(s) from electrode pads that include the pair of electrodes associated with the optimal vector.

In some cases, the recommendation is to administer multiple electrical shocks to electrodes associated with multiple optimal vectors. For instance, the entity may detect multiple optimal vectors. In some cases, the recommendation is to administer a multi-shock therapy (e.g., a DSD therapy) along the multiple optimal vectors.

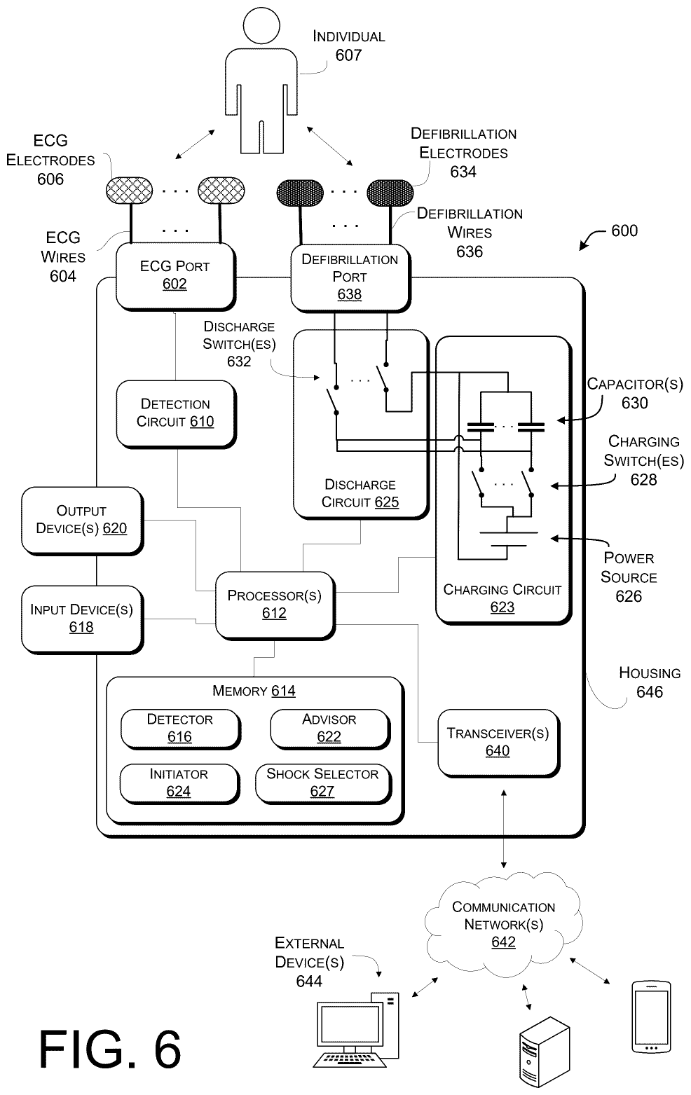

FIG. 6 illustrates an example of an external defibrillator 600 configured to perform various functions described herein. For example, the external defibrillator 600 is the defibrillator 106 described above with reference to FIG. 1.

The external defibrillator 600 includes an electrocardiogram (ECG) port 602 connected to multiple ECG wires 604. In some cases, the ECG wires 604 are removeable from the ECG port 602. For instance, the ECG wires 604 are plugged into the ECG port 602 via connectors. The ECG wires 604 are connected to ECG electrodes 606, respectively. In various implementations, the ECG electrodes 606 are disposed on different locations on an individual 608. A detection circuit 610 (also referred to as a “measurement circuit”) is configured to detect relative voltages between the ECG electrodes 606. These voltages are indicative of the electrical activity of the heart of the individual 608.

In various implementations, the ECG electrodes 606 are in contact with the different locations on the skin of the individual 608. In some examples, a first one of the ECG electrodes 606 is placed on the skin between the heart and right arm of the individual 608, a second one of the ECG electrodes 606 is placed on the skin between the heart and left arm of the individual 608, and a third one of the ECG electrodes 606 is placed on the skin between the heart and a leg (either the left leg or the right leg) of the individual 608. In these examples, the detection circuit 610 is configured to measure the relative voltages between the first, second, and third ECG electrodes 606. Respective pairings of the ECG electrodes 606 are referred to as “leads,” and the voltages between the pairs of ECG electrodes 606 are known as “lead voltages.” In some examples, more than three ECG electrodes 606 are included, such that 5-lead or 12-lead ECG signals are detected by the detection circuit 610.

The detection circuit 610 includes at least one analog circuit, at least one digital circuit, or a combination thereof. The detection circuit 610 receives the analog electrical signals from the ECG electrodes 606, via the ECG port 602 and the ECG wires 604. In some cases, the detection circuit 610 includes one or more analog filters configured to filter noise and/or artifact from the electrical signals. The detection circuit 610 includes an analog-to-digital (ADC) in various examples. The detection circuit 610 generates a digital signal indicative of the analog electrical signals from the ECG electrodes 606. This digital signal can be referred to as an “ECG signal” or an “ECG.”

In some cases, the detection circuit 610 further detects an electrical impedance between at least one pair of the ECG electrodes 606. For example, the detection circuit 610 includes, or otherwise controls, a power source that applies a known voltage (or current) across a pair of the ECG electrodes 606 and detects a resultant current (or voltage) between the pair of the ECG electrodes 606. The impedance is generated based on the applied signal (voltage or current) and the resultant signal (current or voltage). In various cases, the impedance corresponds to respiration of the individual 608, chest compressions performed on the individual 608, and other physiological states of the individual 608. In various examples, the detection circuit 610 includes one or more analog filters configured to filter noise and/or artifact from the resultant signal. The detection circuit 610 generates a digital signal indicative of the impedance using an ADC. This digital signal can be referred to as an “impedance signal” or an “impedance.”

The detection circuit 610 provides the ECG signal and/or the impedance signal one or more processors 612 in the external defibrillator 600. In some implementations, the processor(s) 612 includes a central processing unit (CPU), a graphics processing unit (GPU), both CPU and GPU, or other processing unit or component known in the art.

The processor(s) 612 is operably connected to memory 614. In various implementations, the memory 614 is volatile (such as random-access memory (RAM)), non-volatile (such as read only memory (ROM), flash memory, etc.) or some combination of the two. The memory 614 stores instructions that, when executed by the processor(s) 612, causes the processor(s) 612 to perform various operations. In various examples, the memory 614 stores methods, threads, processes, applications, objects, modules, any other sort of executable instruction, or a combination thereof. In some cases, the memory 614 stores files, databases, or a combination thereof. In some examples, the memory 614 includes, but is not limited to, RAM, ROM, electrically erasable programmable read-only memory (EEPROM), flash memory, or any other memory technology. In some examples, the memory 614 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 612 and/or the external defibrillator 600. In some cases, the memory 614 at least temporarily stores the ECG signal and/or the impedance signal.

In various examples, the memory 614 includes a detector 616, which causes the processor(s) 612 to determine, based on the ECG signal and/or the impedance signal, whether the individual 608 is exhibiting a particular heart rhythm. For instance, the processor(s) 612 determines whether the individual 608 is experiencing a shockable rhythm that is treatable by defibrillation. Examples of shockable rhythms include ventricular fibrillation (VF) and ventricular tachycardia (V-Tach). In some examples, the processor(s) 612 determines whether any of a variety of different rhythms (e.g., asystole, sinus rhythm, atrial fibrillation (AF), etc.) are present in the ECG signal.

The processor(s) 612 is operably connected to one or more input devices 618 and one or more output devices 620. Collectively, the input device(s) 618 and the output device(s) 620 function as an interface between a user and the defibrillator 600. The input device(s) 618 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 620 includes at least one of a display, a speaker, a haptic output device, a printer, or any combination thereof. In various examples, the processor(s) 612 causes a display among the input device(s) 618 to visually output a waveform of the ECG signal and/or the impedance signal. In some implementations, the input device(s) 618 includes one or more touch sensors, the output device(s) 620 includes a display screen, and the touch sensor(s) are integrated with the display screen. Thus, in some cases, the external defibrillator 600 includes a touchscreen configured to receive user input signal(s) and visually output physiological parameters, such as the ECG signal and/or the impedance signal.

In some examples, the memory 614 includes an advisor 622, which, when executed by the processor(s) 612, causes the processor(s) 612 to generate advice and/or control the output device(s) 620 to output the advice to a user (e.g., a rescuer). In some examples, the processor(s) 612 provides, or causes the output device(s) 620 to provide, an instruction to perform CPR on the individual 608. In some cases, the processor(s) 612 evaluates, based on the ECG signal, the impedance signal, or other physiological parameters, CPR being performed on the individual 608 and causes the output device(s) 620 to provide feedback about the CPR in the instruction. According to some examples, the processor(s) 612, upon identifying that a shockable rhythm is present in the ECG signal, causes the output device(s) 620 to output an instruction and/or recommendation to administer a defibrillation shock to the individual 608.

The memory 614 also includes an initiator 624 which, when executed by the processor(s) 612, causes the processor(s) 612 to control other elements of the external defibrillator 600 to administer a defibrillation shock to the individual 608. In some examples, the processor(s) 612 executing the initiator 624 selectively causes the administration of the defibrillation shock based on determining that the individual 608 is exhibiting the shockable rhythm and/or based on an input from a user (received, e.g., by the input device(s) 618. In some cases, the processor(s) 612 causes the defibrillation shock to be output at a particular time, which is determined by the processor(s) 612 based on the ECG signal and/or the impedance signal.

In various cases, the memory 614 includes a shock selector 627 which, when executed by the processor(s) 612, causes the processor(s) 612 to perform operations described herein related to identifying and selecting optimal shock vectors. For instance, the shock selector 627 is configured to cause the defibrillator 600 to output one or more electrical signals (e.g., test shocks) to pairs of the defibrillation electrodes 634 along different shock vectors, and to analyze feedback from the electrical signal(s). Based on the feedback, the shock selector 627 is configured to identify one or more optimal vectors, which can be subsequently used to apply an electrical therapy to the individual 607.

The processor(s) 612 is operably connected to a charging circuit 623 and a discharge circuit 625. In various implementations, the charging circuit 623 includes a power source 626, one or more charging switches 628, and one or more capacitors 630. The power source 626 includes, for instance, a battery. The processor(s) 612 initiates a defibrillation shock by causing the power source 626 to charge at least one capacitor among the capacitor(s) 630. For example, the processor(s) 612 activates at least one of the charging switch(es) 628 in the charging circuit 623 to complete a first circuit connecting the power source 626 and the capacitor to be charged. Then, the processor(s) 612 causes the discharge circuit 625 to discharge energy stored in the charged capacitor across a pair of defibrillation electrodes 634, which are in contact with the individual 608. For example, the processor(s) 612 deactivates the charging switch(es) 628 completing the first circuit between the capacitor(s) 630 and the power source 626 and activates one or more discharge switches 632 completing a second circuit connecting the charged capacitor 630 and at least a portion of the individual 608 disposed between defibrillation electrodes 634. In various cases, more than two defibrillation electrodes 634 are disposed on the skin of the individual 607, which define multiple shock vectors.

The energy is discharged from the defibrillation electrodes 634 in the form of a defibrillation shock. For example, the defibrillation electrodes 634 are connected to the skin of the individual 608 and located at positions on different sides of the heart of the individual 608, such that the defibrillation shock is applied across the heart of the individual 608. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of about 0.015 seconds. In some cases, the defibrillation shock has a multiphasic (e.g., biphasic) waveform. The discharge switch(es) 632 are controlled by the processor(s) 612, for example. In various implementations, the defibrillation electrodes 634 are connected to defibrillation leads 636. The defibrillation wires 636 are connected to a defibrillation port 638, in implementations. According to various examples, the defibrillation wires 636 are removable from the defibrillation port 638. For example, the defibrillation wires 636 are plugged into the defibrillation port 638.

In various implementations, the processor(s) 612 is operably connected to one or more transceivers 640 that transmit and/or receive data over one or more communication networks 642. For example, the transceiver(s) 640 includes a network interface card (NIC), a network adapter, a local area network (LAN) adapter, or a physical, virtual, or logical address to connect to the various external devices and/or systems. In various examples, the transceiver(s) 640 includes any sort of wireless transceivers capable of engaging in wireless communication (e.g., radio frequency (RF) communication). For example, the communication network(s) 642 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 640 includes other wireless modems, such as a modem for engaging in WI-FI®, WIGIG®, WIMAX®, BLUETOOTH®, or infrared communication over the communication network(s) 642.

The defibrillator 600 is configured to transmit and/or receive data (e.g., ECG data, impedance data, data indicative of one or more detected heart rhythms of the individual 608, data indicative of one or more defibrillation shocks administered to the individual 608, etc.) with one or more external devices 644 via the communication network(s) 642. The external devices 644 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 642. In some examples, the external device(s) 644 is located remotely from the defibrillator 600, such as at a remote clinical environment (e.g., a hospital). According to various implementations, the processor(s) 612 causes the transceiver(s) 640 to transmit data to the external device(s) 644. In some cases, the transceiver(s) 640 receives data from the external device(s) 644 and the transceiver(s) 640 provide the received data to the processor(s) 612 for further analysis.

In various implementations, the external defibrillator 600 also includes a housing 646 that at least partially encloses other elements of the external defibrillator 600. For example, the housing 646 encloses the detection circuit 610, the processor(s) 612, the memory 614, the charging circuit 623, the transceiver(s) 640, or any combination thereof. In some cases, the input device(s) 618 and output device(s) 620 extend from an interior space at least partially surrounded by the housing 646 through a wall of the housing 646. In various examples, the housing 646 acts as a barrier to moisture, electrical interference, and/or dust, thereby protecting various components in the external defibrillator 600 from damage.

In some implementations, the external defibrillator 600 is an automated external defibrillator (AED) operated by an untrained user (e.g., a bystander, layperson, etc.) and can be operated in an automatic mode. In automatic mode, the processor(s) 612 automatically identifies a rhythm in the ECG signal, makes a decision whether to administer a defibrillation shock, charges the capacitor(s) 630, discharges the capacitor(s) 630, or any combination thereof. In some cases, the processor(s) 612 controls the output device(s) 620 to output (e.g., display) a simplified user interface to the untrained user. For example, the processor(s) 612 refrains from causing the output device(s) 620 to display a waveform of the ECG signal and/or the impedance signal to the untrained user, to simplify operation of the external defibrillator 600.

In some examples, the external defibrillator 600 is a monitor-defibrillator utilized by a trained user (e.g., a clinician, an emergency responder, etc.) and can be operated in a manual mode or the automatic mode. When the external defibrillator 600 operates in manual mode, the processor(s) 612 cause the output device(s) 620 to display a variety of information that may be relevant to the trained user, such as waveforms indicating the ECG data and/or impedance data, notifications about detected heart rhythms, and the like.

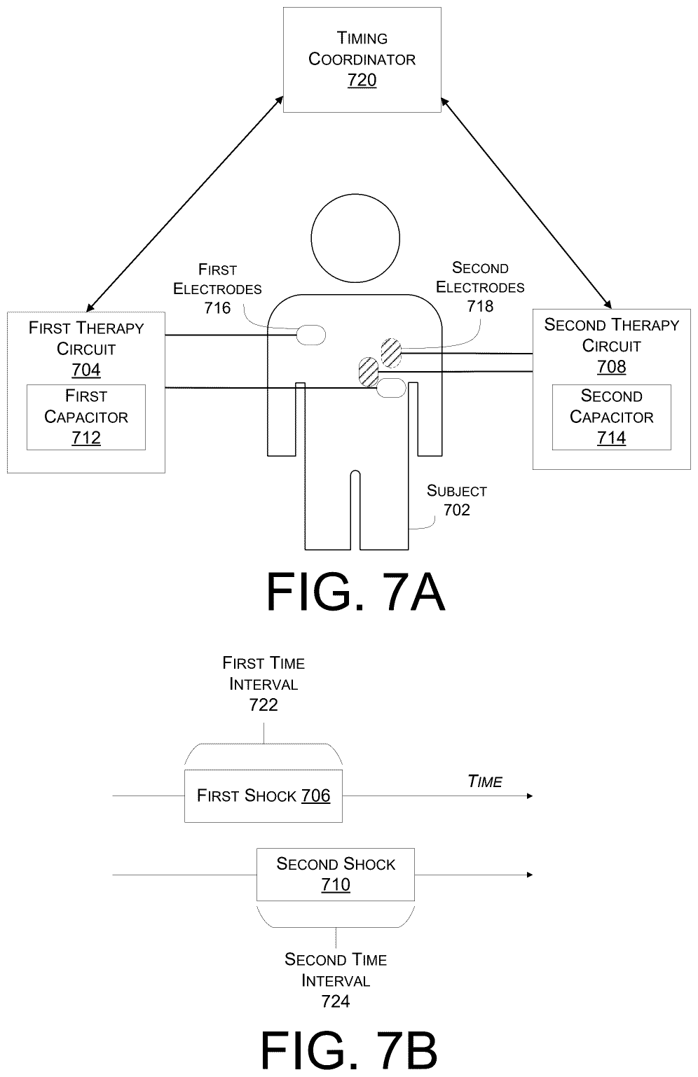

FIGS. 7A and 7B illustrate examples of environments and timing related to administering a multi-shock therapy, such as a DSD therapy. FIG. 7A shows an environment configured to administer the multi-shock therapy. FIG. 7B shows a timing relationship of multiple shocks administered in the multi-shock therapy.

In various cases, a subject 702 has a medical condition that is treatable by defibrillation. For example, the subject 702 may have a shockable cardiac arrhythmia, such as VF or pulseless VT. In some cases, however, conventional defibrillation therapies do not resolve the medical condition. For example, the subject 702 may be experiencing VF that does not resolve after the administration of a single biphasic electrical shock administered by a defibrillator. For instance, the subject 702 may have refractory VF.

In various implementations of the present disclosure, the medical condition of the subject 702 is treatable by administration of a multi-shock therapy. Specifically, a first therapy circuit 704 is configured to output a first shock 706 to the subject 702 and a second therapy circuit 708 is configured to output a second shock 710 to the subject 702. In various cases, the first shock 706 and the second shock 710 temporally overlap in time, at least partially. For example, a start time of the second shock 710 occurs after the start time of the first shock 706, but the start time of the second shock 710 occurs before the end time of the first shock 706. In various cases, the first shock 706 is a biphasic shock and/or the second shock 710 is a biphasic shock. In some implementations, the first shock 706 is a monophasic shock and/or the second shock 710 is a biphasic shock. In some cases, the first shock 706 has a shorter duration and/or lower voltage amplitude than the second shock 710.

The first therapy circuit 704 outputs the first shock 706 by discharging a first capacitor 712. Similarly, the second therapy circuit 708 outputs the second shock 710 by discharging a second capacitor 714. In various implementations, one or more power sources are configured to charge the first capacitor 712 and/or the second capacitor 714 prior to discharge. In various cases, the first therapy circuit 704 includes a first H-bridge circuit including the first capacitor 712 and/or the second therapy circuit 708 includes a second H-bridge circuit including the second capacitor 714. The first H-bridge circuit and the second H-bridge circuit are configured to output the first shock 706 and the second shock 710 as biphasic shocks, for instance, via sequential activation of switches in the first H-bridge circuit and the second H-bridge circuit.

The first therapy circuit 704 is configured to output the first shock 706 to first electrodes 716. The second therapy circuit 708 is configured to output the second shock 710 to second electrodes 718. In various cases, the first electrodes 716 and/or the second electrodes 718 are disposed externally on the skin of the subject 702. For example, the first electrodes 716 and/or the second electrodes 718 are adhered to the skin of the subject 702. In various implementations, the first electrodes 716 and the second electrodes 718 are associated with different shock vectors. For instance, a first shock vector extends between the first electrodes 716 and a second shock vector extends between the second electrodes 718, wherein the first shock vector and the second shock vector are different. For example, the first shock vector may be an anterior-lateral position and the second shock vector may be an anterior-posterior position. In various implementations, the first shock vector and the second shock vector both extend through the heart of the subject 702. Although FIG. 7A illustrates the first electrodes 716 as being separate from the second electrodes 718, implementations are not so limited. For example, one electrode may be shared among the first electrodes 716 and the second electrodes 718. In various cases, both the first shock vector and the second shock vector are optimal vectors, as described elsewhere herein.

The first therapy circuit 704 and the second therapy circuit 708 are distributed among one or more devices. In some cases, the first therapy circuit 704 is part of a first external defibrillator and the second therapy circuit 708 is part of a second external defibrillator. For example, the first external defibrillator and the second external defibrillator are both monitor-defibrillators, both AEDs, or a monitor-defibrillator and an AED. In some cases, the first therapy circuit 704 or the second therapy circuit 708 is integrated into an accessory device without monitoring capabilities, and which is solely designed to output electrical shocks upon receiving an input signal from a separate device. For example, the accessory device may lack, or be disconnected from, one or more sensors configured to identify one or more physiological parameters of the subject 702. In some cases, the accessory device lacks a display, speaker, or other user interface device. In some implementations, the first therapy circuit 704 and the second therapy circuit 708 are integrated into the same device, such as the same monitor-defibrillator.

According to some cases, the one or more devices including the therapy circuit 704 and the second therapy circuit 708 are configured to output the first shock 706 and the second shock 710 in response to receiving a user input signal associated with the multi-shock therapy. For example, the device(s) may output the multi-shock therapy upon receiving two consecutive presses of a shock button. In some cases, the device(s) are configured to output a single-shock therapy in response to receiving a single press of the same shock button.

Optionally, a timing coordinator 720 is configured to cause the first therapy circuit 704 to output the first shock 706 during a first time interval 722 and/or to cause the second therapy circuit 708 to output the second shock 710 during the second time interval 724. For example, the timing coordinator 720 outputs one or more signals (e.g., electrical signals, communication signals, etc.) to the first therapy circuit 704 and/or the second therapy circuit 708. Upon receiving the signal(s) from the timing coordinator 720, the first therapy circuit 704 may discharge the first capacitor 712 during the first time interval 722 and/or the second therapy circuit 708 may discharge the second capacitor 714 during the second time interval 724. The timing coordinator 720 can be implemented in hardware (e.g., a circuit), software (e.g., instructions executed by at least one processor), or a combination thereof. In some cases, the timing coordinator 720 is a standalone device. Examples of standalone timing devices that can serve as the timing coordinator 720 are described in U.S. Pat. No. 10,981,014, which is incorporated by reference herein in its entirety. In some examples, the timing coordinator 720 is integrated into the same device as the first therapy circuit 704 and/or the second therapy circuit 708.

Various timing relationships between the first time interval 722 and the second time interval 724 can be implemented according to various implementations of the present disclosure. In some cases, a delay between the start times (i.e., the leading edges) of the first time interval 722 and the second time interval 724 is in a range of 0 and 250 milliseconds (ms). In some cases, the delay between the start times of the first time interval 722 and the second time interval 724 is in a range of −250 and 0 ms. Although FIG. 7B illustrates the first time interval 722 and the second time interval 724 as having equivalent durations, implementations are not so limited. For example, the first time interval 722 may be longer or shorter than the second time interval 724. Various timing relationships are described in U.S. Pat. No. 10,702,701, which is incorporated by reference herein in its entirety.

In various cases, the timing coordinator 720 is configured to detect the first shock 706 and may cause the second therapy circuit 708 to output the second shock 710 in response. For example, the timing coordinator 720 may detect a signal indicative of the discharge of the first shock 706 and may output a signal that causes the second therapy circuit 708 to discharge the second shock 710. In some examples, the timing coordinator 720 is inductively coupled with the first therapy circuit 704 and/or the first electrodes 716, which enables the timing coordinator 720 to detect the discharge of the first shock 706. Various techniques for detecting the discharge of a first shock to cause the application of a second shock in multi-shock therapy are described in U.S. Pat. Nos. 10,625,088 and 10,632,320, which are incorporated by reference herein in their entirety.

### EXAMPLE CLAUSES

1. A first external defibrillator, including: a treatment circuit configured to: output a first test shock to a first pair of electrodes configured to be disposed on skin of a subject, the first pair of electrodes being associated with a first vector; and output a second test shock to a second pair of electrodes configured to be disposed on the skin of the subject, the second pair of electrodes being associated with a second vector; and output a first treatment shock, the first treatment shock having a higher energy than the first test shock or the second test shock; a detection circuit configured to: detect an electrocardiogram (ECG) of the subject; and a processor configured to: determine that the first vector is an optimal vector by: identifying a perturbation in a shockable rhythm in the ECG in response to the first test shock, the shockable rhythm including ventricular fibrillation (VF) or pulseless ventricular tachycardia (VT); and determining that the shockable rhythm was continuously present in the ECG in response to the second test shock; in response to determining that the first vector is the optimal vector: cause the treatment circuit to output the first treatment shock to the first pair of electrodes; and cause a second external defibrillator to output a second treatment shock to the subject, the second treatment shock temporally overlapping with the first treatment shock.
2. The first external defibrillator of clause 1, wherein the processor is further configured to determine that the first vector is an optimal vector by: determining that a change in a transthoracic impedance detected between the first pair of electrodes associated with movement of a heart of the subject is greater than a change in a transthoracic impedance detected between the second pair of electrodes associated with the movement of the heart.
3. The first external defibrillator of clause 1 or 2, further including: a button configured to detect two consecutive presses from a user, the two consecutive presses being within a threshold time interval; and an electrode assembly configured to be disposed on skin of the subject, the electrode assembly including: an electrically insulative substrate; an electrically conductive hydrogel; a first electrode disposed between the electrically insulative substrate and the hydrogel, the first electrode being among the first pair of electrodes; a second electrode disposed between the electrically insulative substrate and the hydrogel, the second electrode being among the second pair of electrodes; and an adhesive configured to adhere the electrically insulative substrate to the skin of the subject, wherein the processor is configured to cause the treatment circuit to output the first treatment shock to the first pair of electrodes in response to the button detecting the two consecutive presses from the user.
4. A method, including: identifying feedback from electrical signals output along multiple vectors to multiple electrodes configured to be disposed on skin of a subject; selecting, among the multiple vectors, an optimal vector by analyzing the feedback; identifying, among the multiple electrodes, a first electrode and a second electrode associated with the optimal vector; and in response to identifying the first electrode and the second electrode associated with the optimal vector: outputting a recommendation to administer an electrical shock to the first electrode and the second electrode; or outputting the electrical shock to the first electrode and the second electrode.
5. The method of clause 4, wherein the electrical signals include test shocks, each of the test shocks having a lower energy than the electrical shock.
6. The method of clause 4 or 5, wherein the feedback includes transthoracic impedances along the multiple vectors.
7. The method of any of clauses 4 to 6, wherein selecting, among the multiple vectors, the optimal vector by analyzing the feedback includes: identifying a perturbation in a shockable rhythm indicated by an electrocardiogram (ECG) of the subject in response to an electrical signal among the electrical signals being output along the optimal vector.
8. The method of any of clauses 4 to 7, wherein selecting, among the multiple vectors, the optimal vector by analyzing the feedback includes: predicting, by analyzing the feedback, that greater than a threshold amount of energy from the electrical shock output along the optimal vector would be delivered to a heart of the subject.
9. The method of any of clauses 4 to 8, wherein the multiple vectors include a virtual vector.
10. The method of any of clauses 4 to 9, wherein the optimal vector extends between the first electrode and the second electrode.
11. The method of any of clauses 4 to 10, the optimal vector being a first optimal vector, the electrical shock being a first electrical shock, the method further including: selecting, among the multiple vectors, a second optimal vector; identifying, among the multiple electrodes, a third electrode and a fourth electrode associated with the second optimal vector, wherein the recommendation to administer the first electrical shock to the first electrode and the second electrode is further to administer a second electrical shock to the third electrode and the fourth electrode, and wherein outputting the first electrical shock to the first electrode and the second electrode further includes outputting the second electrical shock to the third electrode and the fourth electrode.
12. The method of clause 11, wherein the first electrical shock temporally overlaps with the second electrical shock.
13. The method of clause 11 or 12, wherein the third electrode or the fourth electrode is the first electrode or the second electrode.
14. A defibrillator, including: a treatment circuit configured to: output electrical signals along multiple vectors to multiple electrodes configured to be disposed on skin of a subject; and output an electrical shock; and a measurement circuit configured to: detect feedback from the electrical signals; and a processor configured to: select, among the multiple vectors, an optimal vector by analyzing the feedback; identify, among the multiple electrodes, a first electrode and a second electrode associated with the optimal vector; and in response to identifying the first electrode and the second electrode associated with the optimal vector: cause the treatment circuit to output the electrical shock to the first electrode and the second electrode.
15. The defibrillator of clause 14, wherein the electrical signals include test shocks, each of the test shocks having a lower energy than the electrical shock.
16. The defibrillator of clause 14 or 15, wherein the feedback includes transthoracic impedances along the multiple vectors.
17. The defibrillator of any of clauses 14 to 16, wherein the processor is configured to select, among the multiple vectors, the optimal vector by analyzing the feedback by: identifying a perturbation in a shockable rhythm indicated by an electrocardiogram (ECG) of the subject in response to an electrical signal among the electrical signals being output along the optimal vector.
18. The defibrillator of any of clauses 14 to 17, wherein the processor is configured to select, among the multiple vectors, the optimal vector by: predicting, by analyzing the feedback, that greater than a threshold amount of energy of the electrical shock output along the optimal vector would be delivered to a heart of the subject.
19. The defibrillator of any of clauses 14 to 18, wherein the optimal vector extends between the first electrode and the second electrode.
20. The defibrillator of any of clauses 14 to 19, the optimal vector being a first optimal vector, the electrical shock being a first electrical shock, wherein the processor is further configured to: select, among the multiple vectors, a second optimal vector; identify, among the multiple electrodes, a third electrode and a fourth electrode associated with the second optimal vector; and cause the treatment circuit to administer a second electrical shock to the third electrode and the fourth electrode.
21. The defibrillator of clause 20, wherein the first electrical shock temporally overlaps with the second electrical shock.
22. An external defibrillator, including: a measurement circuit configured to: detect multiple electrocardiogram (ECG) leads of a subject along multiple vectors associated with multiple pairs of electrodes; a treatment circuit configured to output an electrical shock; and a processor configured to: determine that the ECG leads of the subject are indicative of ventricular fibrillation (VF); identify, among the multiple vectors, an optimal vector among the multiple vectors by identifying that a mean peak of one of the ECG leads associated with the optimal vector is greater than a threshold; in response to identifying the optimal vector: output a recommendation to administer the electrical shock to a pair of electrodes, among the pairs of electrodes, associated with the optimal vector; or cause the treatment circuit to output the electrical shock to the pair of electrodes, among the pairs of electrodes, associated with the optimal vector.
23. The external defibrillator of clause 22, wherein the ECG lead associated with the optimal vector is a first ECG lead among the ECG leads, wherein the measurement circuit is further configured to: determine that a second ECG lead among the ECG leads has a mean peak or frequency that is greater than the threshold; detect a first transthoracic impedance associated with the first ECG lead; detect a second transthoracic impedance associated with the second lead, and wherein the processor is configured to identify, among the multiple vectors, the optimal vector by: determining that the first ECG lead is associated with the optimal vector by determining that the first transthoracic impedance is less than the second transthoracic impedance.
24. The external defibrillator of clause 22 or 23, the electrical shock being a first electrical shock, the pair of electrodes being a first pair of electrodes, wherein the processor is configured to determine that the ECG of the subject is indicative of refractory VF, and wherein the processor is further configured to: in response to determining that the ECG of the subject is indicative of refractory VF, cause the treatment circuit to output a second electrical shock to a second pair of electrodes, the first electrical shock temporally overlapping the second electrical shock.
25. A method, including: detecting a first ECG lead along a first vector associated with a first pair of electrodes; detecting a second ECG lead along a second vector associated with a second pair of electrodes; determining that the first vector is an optimal vector by analyzing the first ECG lead; determining that the second vector is a non-optimal vector by analyzing the second ECG lead; and in response to determining that the first vector is the optimal vector: outputting a recommendation to administer an electrotherapy to the first pair of electrodes; or outputting the electrotherapy to the first pair of electrodes.
26. The method of clause 25, wherein determining that the first vector is the optimal vector by analyzing the first ECG lead includes: determining that a peak of the first ECG lead exceeds a threshold.
27. The method of clause 26, further includes: determining the threshold by determining a peak of the second ECG lead.
28. The method of clause 26 or 27, wherein the peak of the first ECG lead includes an average peak of the first ECG lead.
29. The method of any of clauses 26 to 28, wherein determining that the second vector is the non-optimal vector by analyzing the second ECG lead includes: determining that a peak of the second ECG exceeds a threshold; and determining that a transthoracic impedance between the second pair of electrodes is greater than a transthoracic impedance between the first pair of electrodes.
30. The method of any of clauses 25 to 29, further including: determining that the first ECG is indicative of a shockable arrhythmia.
31. The method of clause 30, wherein the electrotherapy includes: a single-shock therapy including a first electrical shock; or a sequential-shock therapy that includes the first electrical shock and a second electrical shock.
32. The method of clause 31, wherein the shockable arrhythmia includes VF or pulseless VT.
33. The method of clause 31 or 32, wherein the shockable arrhythmia includes atrial fibrillation (AF), and wherein first electrical shock is synchronized with an R-wave or QRS complex of the first ECG.
34. The method of any of clauses 30 to 33, wherein the shockable arrhythmia includes bradycardia, and wherein the electrotherapy includes pacing pulses.
35. A medical device, including: a measurement circuit configured to: detect a first ECG lead along a first vector associated with a first pair of electrodes; detect a second ECG lead along a second vector associated with a second pair of electrodes; and a processor configured to: determine that the first vector is an optimal vector by analyzing the first ECG lead; determine that the second vector is a non-optimal vector by analyzing the second ECG lead; and in response to determining that the first vector is the optimal vector: output a recommendation to administer an electrotherapy to the first pair of electrodes; or cause an electrotherapy to be output the first pair of electrodes.
36. The medical device of clause 35, wherein the processor is configured to determine that the first vector is the optimal vector by analyzing the first ECG lead by: determining that an average peak of the first ECG lead exceeds a threshold.
37. The medical device of clause 35 or 36, wherein the measurement circuit is further configured to: detect a first impedance between the first pair of electrodes; and detect a second impedance between the second pair of electrodes, and wherein the processor is configured to determine that the second vector is the non-optimal vector by: determining that a peak of the second ECG exceeds a threshold; and determining that the second impedance is greater than the first impedance.
38. The medical device of any of clauses 35 to 37, wherein the processor is further configured to: determine that the first ECG is indicative of a shockable arrhythmia.
39. The medical device of clause 38, wherein the electrotherapy includes: a single-shock therapy including a first electrical shock; or a sequential-shock therapy that includes the first electrical shock and a second electrical shock.
40. The medical device of clause 38 or 39, wherein the shockable arrhythmia includes VF or pulseless VT.
41. The medical device of any of clauses 38 to 40, wherein the shockable arrhythmia includes atrial fibrillation (AF), and wherein first electrical shock is synchronized with an R-wave or QRS complex of the first ECG.
42. The medical device of any of clauses 35 to 41, wherein the electrotherapy includes a first electrical shock or a first electrical shock and a second electrical shock, and wherein the medical device further includes: a treatment circuit configured to output the first electrical shock or the first electrical shock and the second electrical shock.
43. The medical device of any of clauses 35 to 42, wherein the first pair of electrodes are coupled with a connector accessory, and wherein the second pair of electrodes are coupled with the connector accessory.
44. A method, including: determining that a subject exhibits a condition; in response to determining that the subject exhibits the condition, outputting a first electrical shock to the subject along a first vector; in response to outputting the first electrical shock, determining that the subject continues to exhibit the condition; and in response to determining that the subject continues to exhibit the condition, outputting a second electrical shock to the subject along a second vector.
45. The method of clause 44, wherein the condition includes VF, VT, AF, or bradycardia.
46. A connector accessory including: a first defibrillator connector configured to be electrically coupled with a first defibrillator; a second defibrillator connector configured to be electrically coupled with a second defibrillator; a hub including: ports configured to be electrically coupled with multiple electrode pads configured to be disposed on skin of a subject; and a circuit configured to: selectively and electrically connect the first defibrillator connector to a first pair of the ports associated with a first vector; and selectively and electrically connect the second defibrillator connector to a second pair of the ports associated with a second vector.
47. The connector accessory of clause 46, wherein the first vector or the second vector is an optimal vector.

### Conclusion

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A first external defibrillator, comprising:
a treatment circuit configured to:
output a first test shock to a first pair of electrodes configured to be disposed on skin of a subject, the first pair of electrodes being associated with a first vector; and
output a second test shock to a second pair of electrodes configured to be disposed on the skin of the subject, the second pair of electrodes being associated with a second vector; and
output a first treatment shock, the first treatment shock having a higher energy than the first test shock or the second test shock;
a detection circuit configured to:
detect an electrocardiogram (ECG) of the subject; and
a processor configured to:
determine that the first vector is an optimal vector by:
identifying a perturbation in a shockable rhythm in the ECG in response to the first test shock, the shockable rhythm comprising ventricular fibrillation (VF) or pulseless ventricular tachycardia (VT); and
determining that the shockable rhythm was continuously present in the ECG in response to the second test shock;
in response to determining that the first vector is the optimal vector:
cause the treatment circuit to output the first treatment shock to the first pair of electrodes; and
cause a second external defibrillator to output a second treatment shock to the subject, the second treatment shock temporally overlapping with the first treatment shock.

2. The first external defibrillator of claim 1, wherein the processor is further configured to determine that the first vector is an optimal vector by:
determining that a change in a transthoracic impedance detected between the first pair of electrodes associated with movement of a heart of the subject is greater than a change in a transthoracic impedance detected between the second pair of electrodes associated with the movement of the heart.

3. The first external defibrillator of claim 1, further comprising:
a button configured to detect two consecutive presses from a user, the two consecutive presses being within a threshold time interval;
an electrode assembly configured to be disposed on skin of the subject, the electrode assembly comprising:
an electrically insulative substrate;
an electrically conductive hydrogel;
a first electrode disposed between the electrically insulative substrate and the hydrogel, the first electrode being among the first pair of electrodes;
a second electrode disposed between the electrically insulative substrate and the hydrogel, the second electrode being among the second pair of electrodes; and
an adhesive configured to adhere the electrically insulative substrate to the skin of the subject,
wherein the processor is configured to cause the treatment circuit to output the first treatment shock to the first pair of electrodes in response to the button detecting the two consecutive presses from the user.

4. A method, comprising:
identifying feedback from electrical signals output along multiple vectors to multiple electrodes configured to be disposed on skin of a subject;
selecting, among the multiple vectors, an optimal vector by analyzing the feedback;
identifying, among the multiple electrodes, a first electrode and a second electrode associated with the optimal vector; and
in response to identifying the first electrode and the second electrode associated with the optimal vector:
outputting a recommendation to administer an electrical shock to the first electrode and the second electrode; or
outputting the electrical shock to the first electrode and the second electrode.

5. The method of claim 4, wherein the electrical signals comprise test shocks, each of the test shocks having a lower energy than the electrical shock.

6. The method of claim 4, wherein the feedback comprises transthoracic impedances along the multiple vectors.

7. The method of claim 4, wherein selecting, among the multiple vectors, the optimal vector by analyzing the feedback comprises:
identifying a perturbation in a shockable rhythm indicated by an electrocardiogram (ECG) of the subject in response to an electrical signal among the electrical signals being output along the optimal vector.

8. The method of claim 4, wherein selecting, among the multiple vectors, the optimal vector by analyzing the feedback comprises:
predicting, by analyzing the feedback, that greater than a threshold amount of energy from the electrical shock output along the optimal vector would be delivered to a heart of the subject.

9. The method of claim 4, wherein the multiple vectors comprise a virtual vector.

10. The method of claim 4, wherein the optimal vector extends between the first electrode and the second electrode.

11. The method of claim 4, the optimal vector being a first optimal vector, the electrical shock being a first electrical shock, the method further comprising:
selecting, among the multiple vectors, a second optimal vector;
identifying, among the multiple electrodes, a third electrode and a fourth electrode associated with the second optimal vector,
wherein the recommendation to administer the first electrical shock to the first electrode and the second electrode is further to administer a second electrical shock to the third electrode and the fourth electrode, and
wherein outputting the first electrical shock to the first electrode and the second electrode further comprises outputting the second electrical shock to the third electrode and the fourth electrode.

12. The method of claim 11, wherein the first electrical shock temporally overlaps with the second electrical shock.

13. The method of claim 11, wherein the third electrode or the fourth electrode is the first electrode or the second electrode.

14. A defibrillator, comprising:
a treatment circuit configured to:
output electrical signals along multiple vectors to multiple electrodes configured to be disposed on skin of a subject; and
output an electrical shock; and
a measurement circuit configured to:
detect feedback from the electrical signals; and
a processor configured to:
select, among the multiple vectors, an optimal vector by analyzing the feedback;
identify, among the multiple electrodes, a first electrode and a second electrode associated with the optimal vector; and
in response to identifying the first electrode and the second electrode associated with the optimal vector:
cause the treatment circuit to output the electrical shock to the first electrode and the second electrode.

15. The defibrillator of claim 14, wherein the electrical signals comprise test shocks, each of the test shocks having a lower energy than the electrical shock.

16. The defibrillator of claim 14, wherein the feedback comprises transthoracic impedances along the multiple vectors.

17. The defibrillator of claim 14, wherein the processor is configured to select, among the multiple vectors, the optimal vector by analyzing the feedback by:
identifying a perturbation in a shockable rhythm indicated by an electrocardiogram (ECG) of the subject in response to an electrical signal among the electrical signals being output along the optimal vector.

18. The defibrillator of claim 14, wherein the processor is configured to select, among the multiple vectors, the optimal vector by:
predicting, by analyzing the feedback, that greater than a threshold amount of energy of the electrical shock output along the optimal vector would be delivered to a heart of the subject.

19. The defibrillator of claim 14, wherein the optimal vector extends between the first electrode and the second electrode.

20. The defibrillator of claim 14, the optimal vector being a first optimal vector, the electrical shock being a first electrical shock, wherein the processor is further configured to:
select, among the multiple vectors, a second optimal vector;
identify, among the multiple electrodes, a third electrode and a fourth electrode associated with the second optimal vector; and
cause the treatment circuit to administer a second electrical shock to the third electrode and the fourth electrode.
